(19) United States
(12) Patent Application Publication
(10) Pub. No.: US 2020/0138081 A1
(43) Pub. Date: May 7, 2020

(54) Biologically active food additive for preventing and treating acute respiratory diseases and flu

(71) Applicant: Parapharm LLC, Penza (RU)

(72) Inventors: Villory I. Strukov, Penza (RU); Mikhail D. Prokhorov, Penza (RU); Olga Jones-Strukova, Grand Prairie, TX (US); Vyacheslav N. Trifonov, Zarechny (RU); Yuliya A. Elistratova, Penza (RU); Konstantin G. Elistratov, Penza (RU); Natalya V. Kurus', Penza (RU); Natalya V. Eremina, Penza (RU); Marina N. Maksimova, Penza (RU); Ramziya T. Galeeva, Penza (RU); Larisa G. Radchenko, Penza (RU); Aleksandr V. Fedorov, Kuznetsk (RU); Evgeny N. Krutyakov, Penza (RU); Tatiana V. Elistratova, Penza (RU); Irina V. Khomyakova, Penza (RU); Galina A. Tolbina, Penza (RU)

(73) Assignee: Parapharm LLC, Penza (RU)

(21) Appl. No.: 16/706,639

(22) Filed: Dec. 6, 2019

Related U.S. Application Data
(63) Continuation-in-part of application No. 14/902,562, filed on Jan. 1, 2016, now abandoned.

(30) Foreign Application Priority Data
Jul. 3, 2013 (RU) 2013130302

(51) Int. Cl. A23L 33/155; A61K 35/63

(52) U.S. Cl. CPC A23L 33/155; A61K 35/63

(57) ABSTRACT

The invention relates to means for preventing and treating conditions associated with acute respiratory diseases and flu, as well as for immunity improvement. The proposed biologically active additive comprises adsorbed drone brood homogenate, the daily dose thereof lying between 75 mg and 500 mg, and vitamin or vitamins of group D and/or their active metabolites, a daily dose thereof being between 25 IE and 50,000 IE.

### CROSS-REFERENCE TO RELATED APPLICATIONS

This is a continuation-in-part application of U.S. application Ser. No. 14/902,562 filed Jan. 1, 2016, which is a U.S. National stage application of International application PCT/RU2014/000487 filed Jul. 3, 2014, which claims priority of Russian application RU2013130302 filed Jul. 3, 2013, all of the above applications being incorporated herein by reference in their entirety.

### FIELD OF THE INVENTION

The present invention relates to products for preventing and treating conditions associated with various forms of cold and virus diseases, as well as for immunity improvement.

### SUMMARY OF THE INVENTION

According to the present invention, a biologically active food additive is proposed for preventing and treating acute respiratory diseases (ARD) and flu. The additive comprises adsorbed homogenate of drone brood (further referred to as the ADBH) together with vitamin or vitamins of D-group and/or their active metabolites. The daily dose of the ADBH is proposed to be between 75 mg and 500 mg, and the daily dose of the vitamin/vitamins lies in the range between 25 IE and 50,000 IE. When used as recommended, the additive prevents or eases the course of ARD and flu.

### DETAILED DESCRIPTION OF THE INVENTION

The present invention proposes using a biologically active additive to prevent and treat ARD and flu, the additive comprising ADBH and vitamin or vitamins of D-group and/or their active metabolites, the daily dose of the ADBH being between 75 mg and 500 mg, and the daily dose of the vitamin/vitamins lying in the range between 25 IE and 50,000IE. The ADBH can be prepared in accordance with teachings disclosed, for example, in the U.S. application Ser. No. 14/345,243 assigned to the assignee of the present invention and incorporated herein by reference in the entirety thereof.

Special mention must be made relating to the structure of the ADBH. In the process of preparing the ADBH, drone brood, known as a perishable product, is mixed with a preserving agent(-s) (conservant(-s)) to prevent same from degrading. The process results in obtaining the final product having shelf time of up to three years. It naturally flows from that that the structure of the ADBH undergoes changes during the process, with no health properties of drone brood lost at that. Therefore, transformation of a perishable product into a persistent one is considered acquisition of a markedly different characteristic thereby.

The invention will be described below in the implementation examples.

### Example 1

A panel study of the preventive efficiency of the proposed additive was conducted for a group, consisting of 22 persons aged from 32 to 68, 9 among them being male and 13—female. The study subjects received, orally or sublingually, one tablet of the additive two times a day during three-month course three times a year with monthly intervals, the object of the study being determining the preventive efficiency of the additive against cold and respiratory diseases. The composition of the additive per one tablet was 100 mg of the ADBH and 300 IE of vitamin D₃.

After a 6-month study in the group, the immunity improvement was clinically observed, which was confirmed by objective examination data: there were no cases of ARD or flu among the study participants during the autumn-winter period.

### Example 2

An additional study was conducted after the above panel study. A group consisted of nine persons, aged from 21 to 59, four of them being male and five female. There was an early case of flu for all the persons involved, and they were administered one tablet of the additive two times a day sublingually for 20 days, the tablet comprising 250 mg of ADBH and 50,000 IE of D_{3.} The duration of the course was deliberately shortened down to 20 days from a month to avoid possible hypervitaminosis D with large doses.

As a result, the persons involved avoided the height of the disease with symptomatology of ARD—absent were high temperature, headache, chills and rhinitis. There were no complications such as bronchitis.

### Example 3

A study was conducted in two groups, each consisting of 11 females, aged from 36 to 64, diagnosed with ARD. Participants in a first group were administered one tablet sublingually of the additive two times a day for a month, a dose of the tablet containing 500 mg of the ADBH and 25 of an active metabolite of vitamin D (25(OH)D—alfacalcidol). Participants in the second group did not receive the additive.

The treatment entailed a milder form of the disease in the first group—absent were a temperature over 38° C., severe nasal blockage and rhinitis. Additionally, the recovery period in seven participants (64±15%) of the first group was 2-5 days shorter that in the second group.

### Example 4

Participated in the study were 61 children from eight to 16 years old. The selection criteria was a reduced level of reference values of immunoglobulines (Ig). The study was aimed at exploring the response of immune status to the additive (150 mg of the ADBH and 100 IE of vitamin D daily). The examination of the children followed the WHO protocol. There was morning fasting blood draw for biochemical examinations. Humoral immune response was evaluated by the level of three classes of immunoglobulines—IgG, IgA, and IgM. Immunoglobulines were detected by an immunoturbidimetric method, using an assay kit of DiaSys Diagnostic Systems (Germany). Statistical processing employed Microsoft Excel 5.1.

### Study Results and Discussing

Variations of the main immunoglobuline classes IgG, IgA, and IgM in blood serum are considered one of objective criteria of immune system responsibility for eliminating both exogenous and endogenous (autoimmune) toxic metabolites and deviations of homeostasis which are potentially harmful therefor.

IgG is the main serum immunoglobuline of a healthy human amounting to 70-75% of the whole immunoglobulin fraction. It is especially active in the secondary immune response and antitoxic immunity.

The serum IgA amounts to 15-20% of the whole immunoglobulin fraction, 80% of IgA molecules being presented in humans in monomers. The most important among IgA forms are secretory IgA. They perform a local protective response to bacterial and viral antigens trying to contact mucous membranes. The secretory IgA, produced by B-cells, selectively bind to bacteria inside the mucous layer preventing the bacteria from adhering to a wall.

The IgM immunoglobulins are the largest antibodies in the blood serum. They are the only antibodies being synthesized even before a child was born. IgM are capable of neutralizing foreign particles, effectively activating complement, triggering response of the body.

Normal immunoglobulin variations are as follows, in g/l: IgA from 0.9 to 4.5; IgG from 8 to 17; IgM for boys from 0.5 to 3.2 and from 0.6 to 3.7 for girls.

All the participants of the examination showed low levels of the reference values of immunoglobulines (lower than 2.5 g/l for IgA). To correct them, one tablet in the morning and one tablet before bed (i.e. two tablets daily) of the additive for 30 days were administered, one tablet comprising 75 mg of the ADBH and 100 IE of D₃.

Table 1 illustrates comparative characterization of humoral immune response (antibody response) for the main immunoglobuline classes IgG, IgA, and IgM before the start and after the completion of the month-long course of the additive.

| | Initially | | After course completion | |
|---|---|---|---|---|
| Indices | Girls, n = 30 | Boys, n = 31 | Girls, n = 27 | Boys, n = 29 |
| IgA, g/l | 1.87 ± 0.2** | 1.69 ± 0.1* | 2.3 ± 0.22* | 1.93 ± 0.1* |
| IgG, g/l | 12.44 ± 0.21 | 12.02 ± 0.68 | 12.78 ± 0.2 | 13.13 ± 0.8 |
| IgM, g/l | 1.78 ± 0.19** | 1.99 ± 0.2** | 2.2 ± 0.21 | 2.26 ± 0.20** |

Notes:
n—number of participants;
significance of differences:
*<0.05;
**<0.01

Conclusion: administration of the additive by one tablet twice a day (150 mg of the ADBH and 200 IE of vitamin D in total) for 30 days showed statistically significant increase of the level of immunoglobulins, testifying the increase of immune status in children.

Resulting from that, the above-identified group of children showed immunity improvement corroborated by objective checkup data: not a single child turned sick with ARD and flu.

### Example 5

The object of the study lied in analyzing comparative effect of main ingredients of the additive (the ADBH and vitamin D, separately) on the immune system of the body by variations of the main classes of immunoglobulins IgG, IgA, and IgM in blood serum. As stated previously, IgG is the main serum immunoglobuline of a healthy human amounting to 70-75% of the whole immunoglobulin fraction. It is especially active in the immune response. The serous IgA makes up to 15-20% of the whole immunoglobulin fraction. The most active form of IgA are secretory IgA responsible for a local protection against bacterial and viral antigens, the protection being achieved by binding to bacteria inside the mucous layer, thus preventing the bacteria from adhering to a wall. The IgM immunoglobulins are capable of neutralizing foreign particles, effectively activating complement, triggering defensive response mechanisms in the body.

29 participants, 37 to 60 years old, residing in the city of Penza and suffering from frequent catarrhal diseases were examined, and low levels of immunoglobulin reference values were uncovered. In view of the task set, immunopotentiation was administered to them. Depending on the method of immune correction, the participants were divided into three groups:1^{st} group—nine persons took a tablet of the ADBH (150 mg) twice a day for 30 days, 2^{nd} group—8 persons took a tablet of vitamin D₃ (500 IE) twice a day for 30 days, and 3^{rd} group—12 persons took a tablet of the proposed additive (150 mg of the ADBH and 500 IE of vitamin D₃) by one tablet twice a day for 30 days. Table 2 shows values of specific antibody response for immunoglobulines IgA, IgG, and IgM, as well as values M±m, before administering and after same.

TABLE 2

| | | Method of correction | | | | | |
|---|---|---|---|---|---|---|---|
| Immune | Stat. | 1^{st} group, | | 2^{nd} group, | | 3^{rd} group, | |
| para- | para- | n = 9 | | n = 8 | | n = 12 | |
| meters | meters | Before | After | Before | After | Before | After |
| IgA | M ± m | 1.88 ± | 1.92 ± | 1.81 ± | 1.93 ± | 1.9 ± | 2.87 ± |
| | | 0.1 | 0.19 | 0.14 | 0.14 | 0.17 | 0.23* |
| IgG | M ± m | 12.3 ± | 12.4 ± | 12.41 ± | 12.27 ± | 12.43 ± | 12.29 ± |
| | | 0.23 | 0.25 | 0.3 | 0.53 | 0.61 | 0.53 |
| IgM | M ± m | 1.83 ± | 1.87 ± | 1.91 ± | 1.82 ± | 1.78 ± | 2.16 ± |
| | | 0.22 | 0.27 | 0.21 | 0.24 | 0.23 | 0.26* |

Notes:
n—number of participants; significance p of differences before the treatment and after: *<0.05; M—mean value, m—error in arithmetic mean.

It follows from the table that the ingredients composing the proposed additive are much less effective than the proposed additive, if they are used separately. To wit, though values of IgA in groups 1 and 2 somewhat increased—from 1.88±0.1 to 1.92±0.19 after a month-long course, this difference is uncertain (p>0.05), whereas in group 3, where the ingredients were administered jointly, the concentration of IgA significantly increased from 1.9±0.17 to 2.87±0.23 (p<0.05). IgM significantly increased in group 3 only where the ADBH and vitamin D were used in combination thereof. In other words, an unexpected result of the combined use of the ingredients dramatically differs from that where the ingredients are employed individually. In catamnesis, nobody in the 3^{rd} group fell ill with ARD during six months after the study was over, whereas both 1^{st} and 2^{nd} groups had those diseased. The result can probably be explained by the presence of an enhancer of one ingredient of the proposed additive in the other which is on full display only when the both ingredients are used jointly.

### Example 6

The object was to study the effect of active metabolites of vitamin D, as exemplified by 25 (OH)D, on the immune status in participants, judging by the level of immunoglobulines IgG, IgA, and IgM. 32 participants in the age range between 38 and 62 (23 females and 9 males) residing in Penza had no acute diseases and were apparently healthy. The immune status was examined before the therapy started and 30 days after that. Depending on the preparation to be received, the participants were divided into three comparable groups: in the 1^{st} group (10 persons), they were administered the ADBH by 150 mg twice a day, in the 2^{nd} group (nine persons)—50 IE of 25 (OH)D twice a day, and in the 3^{rd} group (13 persons)—both ingredients in the same dosage (one tablet containing 150 mg of the ADBH and 50 IE of 25 (OH)D) twice a day. Immunoglobulines were detected by an immunoturbidimetric method, using an assay kit of DiaSys Diagnostic Systems (Germany).

Table 3 presents mean values (M±m) of specific antibody response for main immunoglobulin classes IgG, IgA, and IgM.

TABLE 3
Values of specific antibody response by immunoglobulins IgG, IgA,
and IgM (initial data and that after one-month treatment)

| Im- | | Method of correction | | | | | |
|---|---|---|---|---|---|---|---|
| mune | Stat. | 1^{st} group, | | 2^{nd} group, | | 3^{rd} group, | |
| para- | para- | n = 10 | | n = 9 | | n = 13 | |
| meters | meters | Before | After | Before | After | Before | After |
| IgA | M ± m | 1.77 ± | 1.81 ± | 1.79 ± | 1.98 ± | 1.9 ± | 2.89 ± |
| | | 0.12 | 0.17 | 0.16 | 0.15 | 0.16 | 0.24* |
| IgG | M ± m | 12.1 ± | 12.3 ± | 12.21 ± | 12.39 ± | 12.41 ± | 12.31 ± |
| | | 0.25 | 0.23 | 0.32 | 0.54 | 0.63 | 0.56 |
| IgM | M ± m | 1.93 ± | 1.87 ± | 1.91 ± | 1.82 ± | 1.78 ± | 2.16 ± |
| | | 0.21 | 0.23 | 0.24 | 0.25 | 0.26 | 0.24* |

Notes:
n—number of participants; significance p of differences before the treatment and after: *<0.05; M—mean value, m—error in arithmetic mean.

The table shows that, when used individually, the ADBH and 25(OH)D work worse than when employed jointly. Specifically, though values of IgA in groups 1 and 2 somewhat increased —from 1.79±0.1 to 1.98±0.15 in the 2^{nd} group after a month-long treatment, this difference is uncertain (p>0.05). In the meantime, in group 3, where the ingredients were administered jointly, the concentration of IgA increased significantly, from 1.9±0.16 to 2.89±0.24 (p<0.05). IgM significantly increased in group 3 only where the ADBH and vitamin D were used together with each other. So, an unexpected favorable result of the combined use of the ingredients essentially stands out when compared with that where the ingredients are employed individually. The obtained result can likely be explained by the presence of a fortifier of one ingredient of the proposed additive in the other which comes in full force only when the both ingredients are used together with each other.

### Example 7

The object was to study the effect of active metabolites of vitamin D, such as 25 (OH)D, together with the ADBH, and of vitamin D together with the ADBH, as compared with the joint use of all of the above ingredients—vitamin D, 25 (OH)D metabolite thereof, and the ADBH—on the immune status in participants, judging by the level of immunoglobulines IgG, IgA, and IgM. Taking part in the study were 45 children of the age from 10 to 16 (21 boys and 24 girls), suffering from osteopenia. The children were divided into three groups—A, B and C. In the A group (12 persons), the children were administered tablets of the following composition: 300 IE of vitamin D and 100 mg of ADBH, one tablet twice a day. In the B group (17 persons) they were given tablets of 100 mg of the ADBH and 0.25 mcg of 25 (OH)D, one tablet twice a day. In the group C (16 persons), all the above ingredients were present in a tablet administered to each person twice a day and comprising 100 mg of the ADBH, 300 IE of vitamin D, and 0.25 mcg of 25 (OH)D). For all the groups, the treatment lasted 30 days, the results thereof being presented in Table 4.

TABLE 4
Values of specific antibody response by immunoglobulins IgG, IgA,
and IgM (values of M ± m before and after treatment)

| | | Method of correction | | | | | |
|---|---|---|---|---|---|---|---|
| Immune | Stat. | group A, n = 12 with | | group B, n = 17 with | | | |
| para- | para- | vitamin D | | 25(OH) of vitamin D | | group C, n = 16 | |
| meters | meters | Before | After | Before | After | Before | After |
| IgA | M ± m | 1.86 ± | 2.92 ± | 1.81 ± | 2.99 ± | 1.9 ± | 2.97 ± |
| | | 0.1 | 0.19 | 0.14 | 0.17 | 0.19 | 0.23* |
| IgG | M ± m | 12.3 ± | 13.4 ± | 12.41 ± | 13.27 ± | 13.43 ± | 14.29 ± |
| | | 0.23 | 0.25 | 0.3 | 0.53 | 0.63 | 0.53 |
| IgM | M ± m | 1.83 ± | 1.97 ± | 1.91 ± | 2.22 ± | 1.78 ± | 2.18 ± |
| | | 0.22 | 0.27 | 0.21 | 0.24 | 0.23 | 0.26* |

Notes:
n—number of participants; significance p of differences before the treatment and after: *<0.05; M—mean value, m—error in arithmetic mean.

The Table 4 data demonstrate that, upon comparing immunologic values in the participants before and after the treatment, the state of specific antibody response in all the three groups changed upwards. Immunoglobulines A, G, and M are also on the rise in all groups towards mean reference values thereof, which attests to enhancing the defensive reserves of the body.

The width of the dose range in the proposed additive results from such individual characteristics of recipients as age, diet, lifestyle, race, country of residence, gender, inherited and past illnesses. By evaluating these criteria, individual doses of the ingredients can be selected, and corrections made based on the treatment dynamics. The lower limit of the range is explained by bioactivity of the additive, whereas the upper limit for the ADBH is explained by applicability thereof and for the vitamin(s)—by toxicity thereof. Exceeding the dosage can be toxic.

What we claim is:

1. Biologically active food additive for preventing and treating acute respiratory diseases and flu, comprising adsorbed homogenate of drone brood, a daily dose thereof being between 75 mg and 500 mg, and vitamin or vitamins of group D and/or their active metabolites, a daily dose thereof being between 25 IE and 50,000 IE.
